(19) United States
(12) Patent Application Publication
(10) Pub. No.: US 2017/0197937 A1
(43) Pub. Date: Jul. 13, 2017

(54) NEW CRYSTAL FORM OF PANTOPRAZOLE SODIUM COMPOUND AND PREPARATION METHOD THEREFOR

(71) Applicants: TIANJIN UNIVERSITY, Tianjin (CN); HAINAN LINGKANG PHARMACEUTICAL CO., LTD, Haikou, Hainan (CN)

(72) Inventors: Hongxun HAO, Tianjin (CN); Linggang TAO, Haikou, Hainan (CN); Shen JIANG, Tianjin (CN); Yongli WANG, Tianjin (CN); Jingkang WANG, Tianjin (CN); Jun LV, Haikou, Hainan (CN); Qiuxiang YIN, Tianjin (CN); Baohong HOU, Tianjin (CN); Zhao XU, Tianjin (CN); Chuang XIE, Tianjin (CN); Zhao WANG, Tianjin (CN)

(73) Assignees: TIANJIN UNIVERSITY, Tianjin (CN); HAINAN LINGKANG PHARMACEUTICAL CO., LTD, Haikou, Hainan (CN)

(21) Appl. No.: 15/304,861

(22) PCT Filed: Nov. 20, 2015
(86) PCT No.: PCT/CN2015/095231

§ 371 Date: Oct. 18, 2016

(30) Foreign Application Priority Data
Feb. 12, 2015 (CN) 201510075979.3

(51) Int. Cl. C07D 401/12

(52) U.S. Cl. CPC C07D 401/12; C07B 2200/13

(57) ABSTRACT

A novel crystalline form is defined by using diffraction angle 2θ° of X-ray powder diffraction pattern and characteristic peaks of differential scanning calorimetry (DSC). Pantoprazole Sodium solid is added to an alcohol solvent to form a suspension with a concentration of 0.05˜0.2 g/mL, and then an antioxidant is added to the suspension, completely dissolving the solid at a temperature of 15˜35° C., and a solventing-out agent is dropwise added to the solution under the application of ultrasonic wave, wherein the amount of the solventing-out agent is 3˜10 times (in volume) of the alcohol solvent; followed by cooling the solution down to 0˜5° C., continuing to stir for 1˜3 h, and suction filtrating obtained solid-liquid suspension to provide a novel crystalline form of Pantoprazole Sodium crystal after drying the product to constant weight.

This application is the U.S. national phase of International Application No. PCT/CN2015/095231 Filed on 20 Nov. 2015 which designated the U.S. and claims priority to Chinese Application Nos. number CN201510075979.3 filed on 12 Feb. 2015, the entire contents of each of which are hereby incorporated by reference.

### FIELD OF THE INVENTION

The invention belongs to separation technology in medicine field, and in particular, relates to a novel crystalline form of Pantoprazole Sodium compound and its preparing method.

### PRIOR ART

Pantoprazole Sodium is white or almost white crystalline powder, which is soluble in water or methanol, and is almost insoluble in trichloromethane or ethyl ether. Pantoprazole Sodium has a chemical name of 5-difluoromethoxy-2-[(3,4-dimethoxy-2-pyridinyl)methyl]sulfinyl-1H-benzimidazole sodium salt, the formula is C₁₆H₁₄F₂N₃NaO₄S with a molecular weight of 405.36, and the structure formula is shown in formula (I). As a proton pump inhibitor, Pantoprazole Sodium is suitable for the treatment of acute upper gastrointestinal bleeding such as duodenal ulcer, gastric ulcer, acute gastric mucosal lesions, composite gastric ulcer and so on. The main pharmacological effect is inhibiting the secretion of gastric acid by binding to the proton pump of parietal cell (i.e., H+/K+-ATP enzyme).

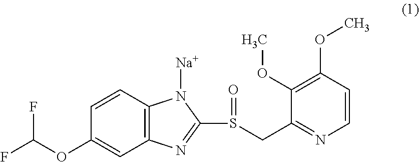

The crystalline form of Pantoprazole Sodium has different crystal structures. WO91/19710 discloses a monohydrate of Pantoprazole Sodium. WO2004100949A discloses complex crystals of Pantoprazole Sodium, including acetone solvates A1-A4, methyl acetate solvates B1-B3, methyl ethyl ketone solvates C1-C2, and diethyl ketone solvate D1. US20040186139A1 discloses an improved method for preparing Pantoprazole Sodium sesquihydrate crystal, including preparation of Pantoprazole Sodium free alkali and a stoichiometric solution of sodium hydroxide in tetrahydrofuran, acetic acid or ethyl acetate, adding an anti-solvent, cooling the solution until a precipitate is formed, and separating the precipitate to give the Pantoprazole Sodium sesquihydrate. US20050245578A1 discloses new crystalline forms of Pantoprazole Sodium monohydrate and single solvate with ketone solvents such as acetone, methyl ethyl ketone, diethyl ketone and methyl isobutyl ketone, which are prepared by contacting Pantoprazole Sodium with the solvents mentioned above. Chinese patent ZL201110228921.X discloses a crystalline hydrate of Pantoprazole Sodium, which is prepared by dissolving Pantoprazole Sodium powder in a mixed solution of water and methanol, and then adding ether to precipitate the crystals.

In summary, Pantoprazole Sodium, as a commonly used drug, is extensively and in-depth researched and some crystalline compounds of Pantoprazole Sodium have been developed. However, the reported crystalline forms of Pantoprazole Sodium in patents are mostly hydrates or solvates with commonly poor thermal stability or low degree of crystallinity, Therefore it is difficult to ensure the reduction or removal of solvent in hydrates or solvates due to the change of external conditions in the subsequent preparation or storage process. Thus, it may cause fluctuations in product quality. To solve the problems described above, the present inventors have found a new crystalline form of Pantoprazole Sodium compound and its corresponding preparing method in the production and research practice.

### DETAILED DESCRIPTION OF THE INVENTION

The present invention discloses a novel crystalline form of Pantoprazole Sodium compound and its preparing method, and the new crystalline form is designated as crystalline form M.

Said novel crystalline form of Pantoprazole Sodium compound has characteristic diffraction peaks expressed in degrees 2θ at 5.62±0.2, 11.62±0.2, 12.16±0.2, 13.80±0.2, 17.18±0.2, 18.80±0.2, 21.72±0.2, 23.44±0.2, 25.16±0.2 and 26.88±0.2 in X-ray powder diffraction pattern, as shown in FIG. 1.

Said novel crystalline form of Pantoprazole Sodium has an endothermic peak at 192±2° C. in differential scanning calorimetry thermogram (DSC), as shown in FIG. 2.

Said novel crystalline form of Pantoprazole Sodium has an appearance of a stubby rod, as shown in FIG. 3.

A method for preparing said novel crystalline form of Pantoprazole Sodium crystal provided in the present invention is as follows:

Adding Pantoprazole Sodium solid to an alcohol solvent to form a suspension with a concentration of 0.050.2 g/mL, and then adding an antioxidant to the suspension, completely dissolving the solid at a temperature of 15˜35° C., and dropwise adding a solventing-out agent to the solution under the application of ultrasonic wave, wherein the amount of the solventing-out agent is 3˜10 times (in volume) of the alcohol solvent; followed by cooling the solution down to 0˜5° C., continuing to stir for 1˜3 h, and suction filtrating obtained solid-liquid suspension to provide a novel crystalline form of Pantoprazole Sodium compound after drying at 50˜60° C.

In said method, the alcohol solvent is selected from one of n-butanol, isobutanol, n-pentanol, isopentanol and n-hexanol or a mixture thereof.

In said method, the antioxidant is selected from one of butylhydroxyanisole (BHA), dibutyl hydroxytoluene (BHT), propyl gallate (PG) and t-butylhydroquinone (TBHQ) or a mixture thereof.

In said method, the amount of the antioxidant is 0.1%˜0.5% in mass of the added Pantoprazole Sodium.

In said method, the solventing-out agent is selected from one of n-heptane, n-hexane, n-pentane, cyclohexane, and n-octane or a mixture thereof.

In said method, the dropwise addition rate of the solventing-out agent is 0.2%˜2% of its volume per minute.

In said method, the ultrasonic wave has a frequency of 20˜50 KHz, and a power of 50˜100 W.

In said method, the cooling rate of the suspension is 0.2˜2 [symbol] /min.

In said method, the drying condition is drying 24˜48 h under normal pressure at a temperature of 50˜60° C.

The feature of the crystalline form M of Pantoprazole Sodium compound provided in the present invention is free of any crystalline solvents, and the melting point of the novel crystalline form is 192±2° C., which is higher than those of traditional crystalline forms. The polymorph, the purity and the appearance do not changed after 15 days in the thermal stability test at conditions of 60° C. and 90% relative humidity, suggesting that the novel crystalline form M has better thermal stability. The product of the novel crystalline form has higher bulk density and fluidity with an appearance of stubby rod. The crystalline product especially has greater advantages in the preparation of formulations of Pantoprazole Sodium tablets, powder and subsequent other forms of such processes, due to the increased stability, fluidity and bulk density. At the same time, the product has better qualities in the process of filtering, washing, drying, packaging and storage due to the improvement of the properties described above. In the meantime, The method for preparing the novel crystalline form of Pantoprazole Sodium compound provided in the present invention is advantageous in its simple and easy-controlled operating conditions, easy-filtered, easy-washed and easy-dried crystal slurry of the product, and the product had a purity of 99% or above with a one-way crystallization process mole yield of 90% or higher.

It is shown on toxic reactions that the novel crystalline form of Pantoprazole Sodium compound provided in the present invention has decreased toxicity than that of existing Pantoprazole Sodium.

### BRIEF DESCRIPTION OF THE DRAWINGS

FIG. 1 shows the X-ray powder diffraction pattern of the novel crystalline form M of Pantoprazole Sodium compound;

FIG. 2 shows the differential scanning calorimetry thermogram of the novel crystalline form M of Pantoprazole Sodium compound;

FIG. 3 shows the microphotograph of the novel crystalline form M of Pantoprazole Sodium compound.

### EMBODIMENTS OF THE INVENTION

The present invention is further illustrated by the following figures and examples. By these illustration, features and advantages of the present invention becomes more clear and more definite.

### EXAMPLE 1

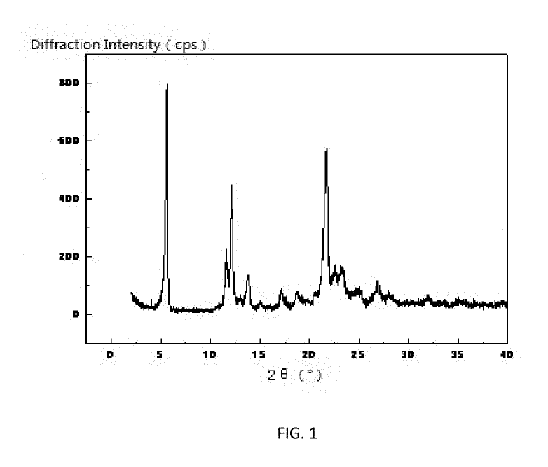

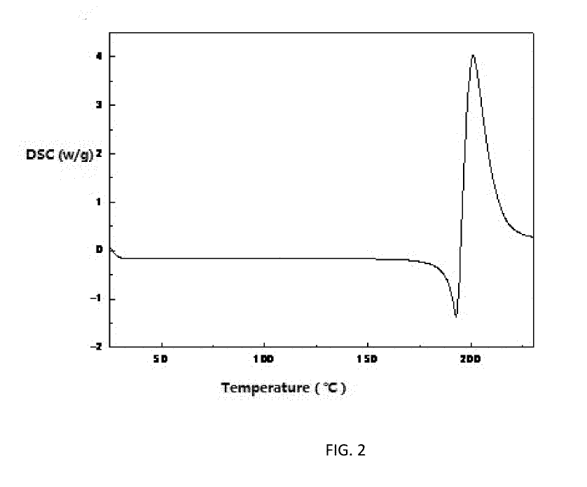

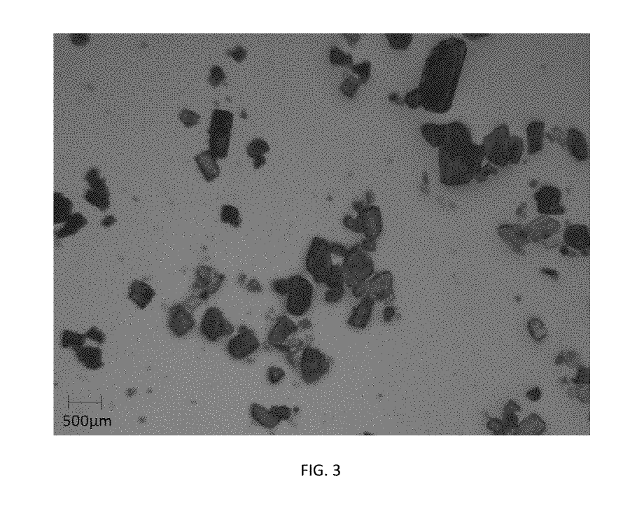

1 g of dried solid Pantoprazole Sodium was added to 20 mL of n-butanol and n-pentanol (1:1, in volume) to form a suspension, and then 0.005 g BHA was added to the suspension under stirring, the suspension was heated to 15 [symbol] to make all the solid dissolved; and 60 mL of cyclohexane was dropwise added to the solution at a speed of 0.12 ml/min under constant temperature to obtain a suspension; meanwhile, a ultrasonic wave with a frequency of 20 KHz, a power of 50 W was applied, followed by cooling the solution down to 0° C. at a rate of 0.2° C./min, continuing to stir for 1 h, vacuum filtrating the crystal slurry, and the residue was dried at 50° C. and under normal pressure for 48 h to constant weight, to obtain crystalline form M of Pantoprazole Sodium product. XRD pattern of the novel crystalline form M was shown in FIG. 1, comprising characteristic peaks (expressed in degrees 2θ) at 5.62, 11.62, 12.16, 13.80, 17.18, 18.80, 21.72, 23.44, 25.16 and 26.88. DSC thermogram of the novel crystalline form M was shown in FIG. 2, comprising an endothermic peak at 192.61° C. The polymorph, the purity and the appearance of the product did not make any change after 15 days at 60° C. and under 90% relative humidity, and the polymorph, the purity and the color of the product did not make any change after 100 days stored at normal temperature and air humidity. The product of the novel crystalline form M had higher bulk density and fluidity with an appearance of stubby rod, as shown in FIG. 3. The product of the novel crystalline form provided by this method had a purity of 99.5% and a one-way crystallization process mole yield of higher than 91.2%.

### EXAMPLE 2

2 g of dried solid Pantoprazole Sodium was added to 30 mL of isopentanol to form a suspension, and then 0.008 g BHA and BHT (1:1, in mass) were added to the suspension under stirring, the suspension was heated to 20 [symbol] to make all the solid dissolved; and 100 mL of n-pentane was dropwise added to the solution at a speed of 1 ml/min under constant temperature to obtain a suspension; meanwhile, a ultrasonic wave with a frequency of 40 KHz, a power of 60 W was applied, followed by cooling the solution down to 2° C. at a rate of 0.5° C./min, continuing to stir for 2 h, vacuum filtrating the crystal slurry, and the residue was dried at 50° C. and under normal pressure for 40 h to constant weight, to obtain crystalline form M of Pantoprazole Sodium product. XRD pattern of the product comprised characteristic peaks (expressed in degrees 2θ) at 5.64, 11.42, 12.18, 13.80, 17.16, 18.82, 21.68, 23.02, 25.18 and 26.98. DSC thermogram of the novel crystalline form M comprised an endothermic peak at 192.12° C. The polymorph, the purity and the appearance of the product did not make any change after 15 days at 60° C. and under 90% relative humidity, and the polymorph, the purity and the color of the product did not make any change after 100 days stored at normal temperature and air humidity. The product of the novel crystalline form M had higher bulk density and fluidity with an appearance of stubby rod. The product of the novel crystalline form provided by this method had a purity of 99.3% and a one-way crystallization process mole yield of 91.5%.

### EXAMPLE 3

2.5 g of dried solid Pantoprazole Sodium was added to 20 mL of n-hexanol to form a suspension, and then 0.01 g PG was added to the suspension under stirring, the suspension was heated to 25 [symbol] to make all the solid dissolved; and 120 mL of n-octane was dropwise added to the solution at a speed of 2 ml/min under constant temperature to obtain a suspension; meanwhile, a ultrasonic wave with a frequency of 30 KHz, a power of 70 W was applied, followed by cooling the solution down to 3° C. at a rate of 1° C./min, continuing to stir for 3 h, vacuum filtrating the crystal slurry, and the residue was dried at 55° C. and under normal pressure for 30 h to constant weight, to obtain crystalline form M of Pantoprazole Sodium product. XRD pattern of the product comprised characteristic peaks (expressed in degrees 2θ) at 5.74, 11.56, 12.26, 13.92, 17.28, 18.88, 21.78, 23.24, 25.22 and 27.08. DSC thermogram of the novel crystalline form M comprised an endothermic peak at 192.80° C. The polymorph, the purity and the appearance of the product did not make any change after 15 days at 60° C. and under 90% relative humidity, and the polymorph, the purity and the color of the product did not make any change after 100 days stored at normal temperature and air humidity. The product of the novel crystalline form M had higher bulk density and fluidity with an appearance of stubby rod. The product of the novel crystalline form provided by this method had a purity of 99.6% and a one-way crystallization process mole yield of 92.1%.

### EXAMPLE 4

4 g of dried solid Pantoprazole Sodium was added to 20 mL of isobutanol to form a suspension, and then 0.004 g TBHQ was added to the suspension under stirring, the suspension was heated to 35 [symbol] to make all the solid dissolved; and 200 mL of n-hexane and n-heptane (1:1, in volume) were dropwise added to the solution at a speed of 1 ml/min under constant temperature to obtain a suspension; meanwhile, a ultrasonic wave with a frequency of 50 KHz, a power of 100 W was applied, followed by cooling the solution down to 5° C. at a rate of 2° C./min, continuing to stir for 4 h, vacuum filtrating the crystal slurry, and the residue was dried at 60° C. and under normal pressure for 24 h to constant weight, to obtain crystalline form M of Pantoprazole Sodium product. XRD pattern of the product comprised characteristic peaks (expressed in degrees 2θ) at 5.62, 11.42, 12.14, 13.80, 17.12, 18.70, 21.64, 23.40, 25.20 and 26.84. DSC thermogram of the novel crystalline form M comprised an endothermic peak at 192.46° C. The polymorph, the purity and the appearance of the product did not make any change after 15 days at 60° C. and under 90% relative humidity, and the polymorph, the purity and the color of the product did not make any change after 100 days stored at normal temperature and air humidity. The product of the novel crystalline form M had higher bulk density and fluidity with an appearance of stubby rod. The product of the novel crystalline form provided by this method had a purity of 99.7% and a one-way crystallization process mole yield of 91.8%.

### Toxic Reaction Tests

The novel crystalline form of Pantoprazole Sodium compound of the present invention was tested by toxic reaction tests shown as follows (taking the crystalline form of Pantoprazole Sodium obtained in Example 1 for example):

The median lethal dose method was taken for the acute toxicity test in mice. 50 ICR mice met experimental requirements were selected and randomly divided into 5 dose groups by weight, 10 animals each group with males and females half and half. The doses were set to 600, 492, 403.4, 330.8 and 271.3 mg/kg. The concentration of Pantoprazole Sodium is 30, 24.6, 20.2, 16.5 and 13.6 mg/mL, administered as intravenous injection. Toxic reactions were observed during administration and after administration in mice. As a result, animals have phenomena of struggling, shortness of breath, limbs limp, difficulty breathing, and severe to death during administration, the LD₅₀ of the Pantoprazole Sodium is 391.52 mg/kg.

15 Beagle dogs were used for acute toxicity test, and given different doses of Pantoprazole Sodium of 44, 67, 100, 150 and 225 mg/kg. 3 animals belongs to each group and toxic reactions were observed after administration in each group. As a result, the dogs expressed as tremors, increased salivation, limp, vomiting, and death after 20 min at the dose of 150 mg/kg. The LD50 is 70 mg/kg. The Pantoprazole Sodium was divided into 5, 3 and 1 g/kg groups by high, medium and low doses. The test period is 13 weeks. 80 healthy Wistar rats with males and females half and half were randomly divided into four groups, and were gavaged once a day regularly, drug withdrawal 2 weeks after administration 13 weeks, and then taken conventional toxicology observations and index detection. As a result, the medium- and low-dose groups had no significant difference occurred; the high-dose group had granulocyte (GR) significantly reduced and alanine aminotransferase (ALT) significantly increased after administration. Histopathological examination showed that liver cell degeneration occurred in one rat. No obvious abnormality occurred in each dose groups of every index after drug withdrawal. The dose of nontoxic response of Pantoprazole Sodium in long-term toxicity test in rats is 3 g·kg⁻¹.

Referring to the novel crystalline form of Pantoprazole Sodium compound and its preparation method which are disclosed and provided in the present invention, with using the present invention for reference, the person skilled in the art could make it implemented by altering materials and process parameter properly. Method and product of the present invention has already been illustrated by preferable embodiments, it will be apparent for related technicians to make changes, modifications and combinations according to the method and product provided by the present invention to achieve technology realization in the present invention, without deviating from the content, spirit and scope of the present disclosure. Especially, all of the similar replacements and modifications are obvious for those skilled in the art, which will be seen to fall within the spirit, scope and content of the present invention.

What is claimed is:

1. A crystalline form of Pantoprazole Sodium crystal, characterized in that, it has an X-ray powder diffraction pattern comprising characteristic diffraction peaks expressed in degrees 2θ at 5.62±0.2, 11.62±0.2, 12.16±0.2, 13.80±0.2, 17.18±0.2, 18.80±0.2, 21.72±0.2, 23.44±0.2, 25.16±0.2 and 26.88±0.2.

2. The crystalline form according to claim 1, characterized in that, it has a differential scanning calorimetry thermogram (DSC) comprising an endothermic peak at 192±2 [symbol] .

3. A method for preparing the crystalline form of Pantoprazole Sodium crystal according to claim 1 or claim 2, characterized in that: adding Pantoprazole Sodium solid to an alcohol solvent to form a suspension with a concentration of 0.05˜0.2 g/mL, and then adding an antioxidant to the suspension, completely dissolving the solid at a temperature of 15˜35 [symbol] , and dropwise adding a solventing-out agent to the solution under the application of ultrasonic wave, wherein the amount of the solventing-out agent is 3˜10 times (in volume) of the alcohol solvent; followed by cooling the solution down to 0˜5° C., continuing to stir for 1˜3 h, and suction filtrating obtained solid-liquid suspension to provide a novel crystalline form product of Pantoprazole Sodium compound after drying at 50˜60° C.

4. The method according to claim 3, characterized in that, the alcohol solvent is selected from one of n-butanol, isobutanol, n-pentanol, isopentanol and n-hexanol or a mixture thereof.

5. The method according to claim 3, characterized in that, said antioxidant is selected from one of butylhydroxyanisole, dibutyl hydroxytoluene, propyl gallate and t-butylhydroquinone or a mixture thereof, and the amount of the antioxidant is 0.1%˜0.5% in mass of the added Pantoprazole Sodium.

6. The method according to claim 3, characterized in that, the solventing-out agent is selected from one of n-heptane, n-hexane, n-pentane, cyclohexane, n-octane or a mixture thereof, and the dropwise addition rate of the solventing-out agent is 0.2%˜2% of its volume per minute.

7. The method according to claim 3, characterized in that, ultrasonic wave is introduced during crystallization, and the ultrasonic frequency is 20˜50 KHz and the power is 50˜100 W.

8. The method according to claim 3, characterized in that, the drying condition lasts 24˜48 h under normal pressure at a temperature of 50˜60 [symbol] .

9. The method according to claim 3, characterized in that, the cooling rate of the suspension is 0.2˜2 [symbol] /min.

10. A process for using the novel crystalline form of Pantoprazole Sodium as a proton pump inhibitor, suitable for dosage forms of tablet and powder-injection formulations.
